(19) United States
(12) Patent Application Publication
(10) Pub. No.: US 2026/0283799 A1
(43) Pub. Date: Sep. 24, 2026

(54) TRANSCATHETER EDGE-TO-EDGE REPAIR IMPLANT WITH USER-SELECTABLE STIFFNESS

(71) Applicant: Evalve, Inc., Santa Clara, CA (US)

(72) Inventors: Chad J. Abunassar, Alameda, CA (US); Manish Gada, San Jose, CA (US); Joanna M. Ignacio, Dublin, CA (US); Saurabh Datta, Pleasanton, CA (US); Matthew G. Jew, San Francisco, CA (US); Anna Maria Snell, Belmont, CA (US); Michael Shin, Pleasanton, CA (US); Gabriel Gonzales, Milpitas, CA (US)

(73) Assignee: Evalve, Inc., Santa Clara, CA (US)

(21) Appl. No.: 19/570,773

(22) Filed: Mar. 18, 2026

Related U.S. Application Data
(60) Provisional application No. 63/774,196, filed on Mar. 19, 2025.

(51) Int. Cl. A61F 2/24

(52) U.S. Cl. CPC A61F 2/246; A61F 2/2466; A61F 2220/0008; A61F 2230/0093; A61F 2250/0012

(57) ABSTRACT

A fixation device includes a primary unit having pair of distal elements and a pair of proximal elements, the pair of distal elements and the pair of proximal elements being configured to retain tissue, a secondary reinforcement coupled to the primary unit and transitionable between a first position spaced away from the primary unit and a second position nested about the primary unit, and a deployment shaft configured to actuate the secondary reinforcement between the first position and the second position. A method of treating a native valve includes providing a fixation device including a primary unit having a pair of distal elements and a pair of proximal elements configured to retain tissue, and a secondary reinforcement, and transitioning the secondary reinforcement from a first position to a second position to increase a stiffness of the fixation device.

### CROSS-REFERENCE TO RELATED APPLICATIONS

This application claims the benefit of the filing date of United States Provisional Patent Application No. 63/774,196, filed Mar. 19, 2025, the disclosure of which is hereby incorporated herein by reference.

### BACKGROUND

The cardiac cycle is divided into two phases—diastole and systole. Diastole is generally characterized by the muscular relaxation of the heart and the filling of its chambers with blood. On the other hand, systole is generally characterized by the muscular contraction of the ventricles which pumps blood from the ventricles to the arteries. During ventricular systole, ventricular pressure increases relative to atrial pressure resulting in the closure of the mitral valve and the tricuspid valve. The mitral valve separates the left atrium from the left ventricle, and the tricuspid valve separates the right atrium from the right ventricle. These valves operate as check valves preventing blood from flowing back into the atria during ventricular contraction. However, valvular insufficiency may appear in one or both of these valves which may result in a regurgitative flow back into the atrium across the effected valve. Such regurgitative flow can be in the form of mitral valve regurgitation (“MVR”) and/or tricuspid valve regurgitation (“TVR”). Left untreated, MVR and TVR can lead to severe health consequences, such as progressive heart failure, cardiac arrythmias, pulmonary hypertension, stroke, and endocarditis, to name a few.

MVR and TVR can have a variety of etiologies which typically fall into the categories of degenerative (primary) and functional (secondary) regurgitation. Degenerative valve regurgitation principally occurs due to abnormalities or degeneration of the valve apparatus, such as the valve leaflets, valve annulus, chordae tendineae, and/or papillary muscles. One example of a degenerative valve condition is mitral valve prolapse. Functional valve regurgitation is often a secondary condition that arises from underlying heart conditions or diseases that affect the structure or function of the heart. Examples of conditions that can result in functional regurgitation include dilated cardiomyopathy, ischemic heart disease, pulmonary hypertension, and heart failure. Regardless of the underlying condition precipitating the regurgitative flow, the primary mechanism by which regurgitation occurs is the failure of the valve leaflets to properly and completely seal or coapt during systole which allows a jet of blood to flow back into the atrium between the effected leaflets.

Treatment options for MVR and TVR generally include Guideline-Directed Medical Therapy (“GDMT”), valve replacement, and valve repair. GDMT usually involves the administration of a combination of drugs that treat an underlying heart condition. Valve replacement and repair may include open-heart surgical options and catheter-based options. Catheter-based repair procedures are sometimes referred to as transcatheter edge-to-edge repair (“TEER”).

### BRIEF SUMMARY OF THE DISCLOSURE

In some examples, a fixation device includes a primary unit having pair of distal elements and a pair of proximal elements, the pair of distal elements and the pair of proximal elements being configured to retain tissue, a secondary reinforcement coupled to the primary unit and transitionable between a first position spaced away from the primary unit and a second position nested about the primary unit, and a deployment shaft configured to actuate the secondary reinforcement between the first position and the second position.

In some examples, a method of treating a native valve includes providing a fixation device including a primary unit having a pair of distal elements and a pair of proximal elements configured to retain tissue, and a secondary reinforcement, and transitioning the secondary reinforcement from a first position to a second position to increase a stiffness of the fixation device.

### BRIEF DESCRIPTION OF THE DRAWINGS

FIG. 1A is a cross-sectional representation of a heart illustrating its four valves.

FIG. 1B is a cross-sectional representation of a heart illustrating the left ventricle and left atrium during systole.

FIG. 2A is a schematic view of a mitral valve during normal coaptation.

FIG. 2B is a schematic view of a mitral valve during regurgitate coaptation.

FIGS. 3A and 3B are schematic views of a fixation device according to an embodiment of the present disclosure grasping leaflets of a mitral valve.

FIG. 4A is a perspective view of a fixation device according to another embodiment of the present disclosure.

FIG. 4B is a perspective view of the fixation device of FIG. 4A including a covering.

FIG. 5A is a perspective view of a gripping device of the of the fixation device of FIG. 4A according to an embodiment of the present disclosure.

FIG. 5B is an elevational view of the gripping device of FIG. 5A.

FIGS. 6A-6B, 7A-7B, 8A-8B, 9A-9B and 10A-10C illustrate the fixation device of FIG. 4A in various possible positions during introduction and placement of the device within a mammalian body to perform a therapeutic procedure.

FIGS. 11A-11D illustrate an embodiment of a fixation device having a secondary reinforcement.

FIG. 12 illustrates another embodiment of a fixation device.

FIGS. 13A-13B illustrate an embodiment of a fixation device similar to that of FIG. 12 with a secondary reinforcement.

FIGS. 13C-13D illustrate another embodiment of a fixation device with a secondary reinforcement.

FIG. 14A illustrates a top schematic view showing a potential difference in size between a primary unit and a secondary reinforcement of a fixation device.

FIG. 14B-14C illustrate another example of a secondary reinforcement.

FIG. 15 illustrates another embodiment of a fixation device having an embedded secondary reinforcement.

FIG. 16 is a flowchart showing one example of a method according the present disclosure.

### DETAILED DESCRIPTION

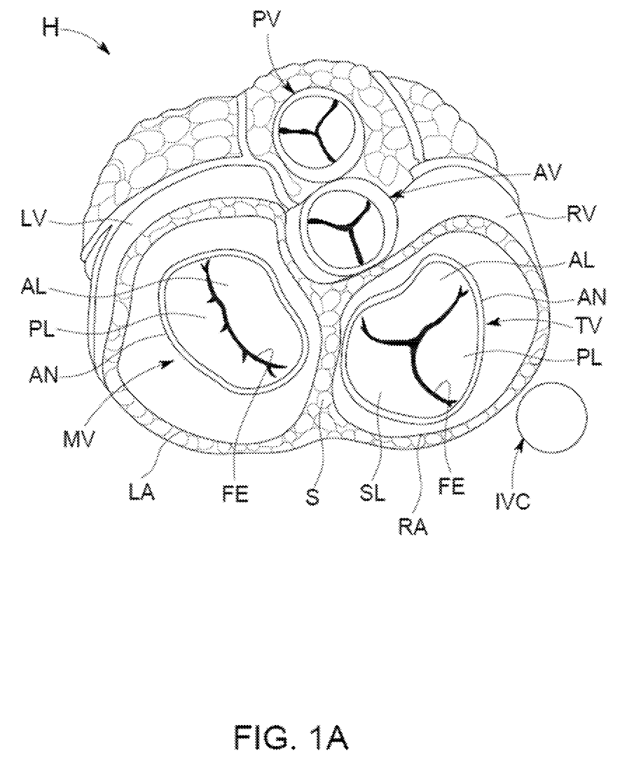

The valves of a normal heart H are illustrated in FIGS. 1A and 1B. These valves include the mitral valve MV, the tricuspid valve TV, the aortic valve AV, and the pulmonary valve PV. The mitral valve MV separates the left atrium LA and the left ventricle LV, and the tricuspid valve TV separates the right atrium RA and the right ventricle RV. The mitral valve MV and the tricuspid valve TV are sometimes referred to as the atrioventricular valves. The mitral valve MV is a bicuspid valve in that it has two leaflets referred to as the posterior leaflet PL and the anterior leaflet AL. The tricuspid valve TV typically has three leaflets referred to as the anterior leaflet AL, the posterior leaflet PL, and the septal leaflet SL. However, studies have shown that, although the TV is typically composed of three leaflets of unequal size, in many cases, two or more than three leaflets may be present as anatomic variants in healthy subjects. Thus, reference herein to the tricuspid valve TV should be understood to refer to the atrioventricular valve located between the right atrium RA and right ventricle RV regardless of the number of leaflets be it two, three, or more than three leaflets. However, exemplary embodiments discussed herein refer to the usual anatomic structure of the tricuspid valve TV that includes three leaflets.

As illustrated in FIG. 1B, the anterior leaflet AL and posterior leaflet PL of the mitral valve MV extend from a valve annulus AN to respective free edges FE. The free edges FE are secured to the lower portions of the left ventricle LV through chordae tendineae CT (referred to hereinafter as the chordae). The chordae CT include a plurality of branching tendons that are attached to papillary muscles PM at the lower portions of the left ventricle LV and extend upwardly to the lower surfaces of each of the valve leaflets where they are attached. The three leaflets of the tricuspid valve TV similarly extend from a valve annulus AN to respective free edges FE which are secured via chordae to the papillary muscles of the right ventricle RV.

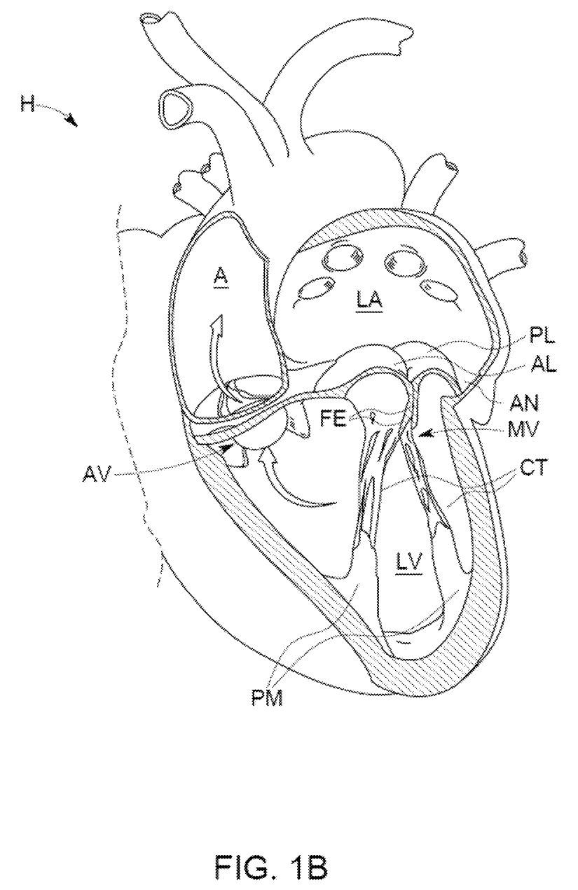

The mitral valve MV depicted in FIGS. 1B and 2A illustrate the proper functioning of an atrioventricular valve during ventricular systole. As the ventricles contract, the free edges FE of adjacent leaflets LF meet along a line of coaptation LOC. The joinder of the leaflets LF at this line of coaptation LOC seals off the ventricle from the atrium and prevents the back flow of blood or “regurgitation” from entering into the atrium. Thus, with the right atrium RA and left atrium LA respectively sealed off by the mitral valve MV and tricuspid valve TV, blood in the left ventricle LV can only flow through the aortic valve AV to the body, and blood in the right ventricle RV can only flow through the pulmonary valve PV to the lungs.

A number of structural defects in the heart H can cause mitral valve regurgitation (“MVR”) and/or tricuspid valve regurgitation (“TVR”). MVR and TVR occur when their respective leaflets LF do not close properly allowing leakage from the ventricle into the atrium. The mitral valve MV depicted in FIG. 2B illustrates valvular insufficiency of an atrioventricular valve resulting in regurgitation. In the depicted example, an enlargement of the heart H may cause the valve annulus AN to become enlarged, making it impossible for the free edges FE of the valve leaflets LF to meet during systole. This may result in a gap G between the leaflets LF which allows blood to leak through the valve. In another example, ruptured or elongated chordae CT can cause a valve leaflet LF to prolapse at least due to inadequate tension transmitted to the leaflet via the chordae CT. While an adjacent leaflet LF may maintain a normal profile, the prolapsing leaflets LF may flail about preventing the proper joinder between the leaflets LF resulting in leakage into the atrium. In a further example, regurgitation can occur in patients who have suffered ischemic heart disease which may result in weak ventricular contractions insufficient to effect proper closure.

The present disclosure describes exemplary systems, devices, and methods for percutaneously repairing a valve to treat cardiac valve regurgitation, particularly MVR and TVR. When referring to such disclosed systems, devices, and methods, the term "proximal" (P) shall mean closer to the user or in a direction toward a device to be manipulated by the user outside the patient’s body, and the term "distal" (D) shall mean more distant from the user or in a direction toward a device that is positioned at the treatment site within the patient’s body (e.g., fixation device 112). With respect to the mitral valve and tricuspid valve, “proximal” shall refer to the atrial or upstream side of the valve leaflets, and “distal” shall refer to the ventricular or downstream side of the valve leaflets.

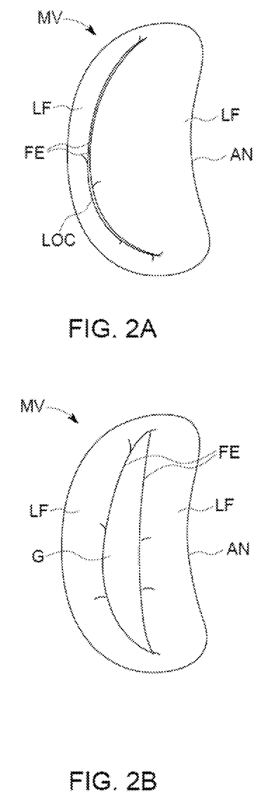

FIGS. 3A and 3B depict a fixation device 12, according to an embodiment of the present disclosure, grasping leaflets LF of an atrioventricular valve, which is illustrated as a mitral valve MV. Fixation device 12 may be releasably coupled to a distal end of a shaft 11 of a delivery system (e.g., delivery system 100) to form an interventional tool 10. Fixation device 12 may include distal elements 20 (also referred to herein as fixation elements) and proximal elements 40 (also referred to herein as gripping elements). Distal and proximal elements 20, 40 may be moveable relative to each other and may protrude radially outward relative to a longitudinal axis A1 of fixation device 12. As shown in FIG. 3A, fixation device 12 may be positionable on opposite sides of adjacent leaflets LF of the valve so as to capture or retain the leaflets LF therebetween. In this regard, proximal elements 40 may be positioned at a proximal side of the valve leaflets LF, and distal elements 20 may be positioned on a distal side of the valve leaflets LF. Proximal elements 40 may be made from cobalt chromium, nitinol, or stainless steel, for example, and distal elements 20 may be made from cobalt chromium or stainless steel, for example.

Fixation device 12 may be releasably coupled to shaft 11 such that it can be detached and left behind as an implant to hold the leaflets LF together in the coapted position. In this regard, fixation device 12 may be delivered to a target valve percutaneously using any one of a number of different approaches, such as via a transfemoral, a transapical, or a transjugular approach, for example. Thus, in one example of treating MVR, fixation device 12 may be delivered to the deficient mitral valve MV using a transfemoral approach in which fixation device 12 is guided through the inferior vena cava IVC (see FIG. 1A), across the interatrial septum S, and into left atrium LA where fixation device 12 is advanced into the mitral valve MV. Also, in one example of treating TVR, fixation device 12 may be guided transfemorally through the inferior vena cava IVC to the right atrium RA where fixation device 12 is advanced to a desired position within the tricuspid valve TV.

FIG. 3B is an atrial-side view of fixation device 12 in one example of a desired orientation in relation to adjacent leaflets LF of an atrioventricular valve, such as the depicted mitral valve MV. The distal and proximal elements 20, 40 are positioned to be substantially perpendicular to the line of coaptation LOC. Thus, in the case of a mitral valve MV, fixation device 12 may be oriented perpendicular (+/- 5 degrees) to a line of coaptation LOC between the posterior leaflet PL and anterior leaflet AL, and in the case of a tricuspid valve TV, fixation device 12 may be positioned perpendicular (+/- 5 degrees) to a line of coaptation between the septal leaflet SL and the anterior leaflet AL, the septal leaflet SL and the posterior leaflet PL, or the anterior leaflet AL and the posterior leaflet PL, for example. Device 12 may be moved roughly along the line of coaptation LOC to the location of regurgitation. The leaflets LF may be held in place so that, during diastole, the leaflets LF remain in position between elements 20, 40 surrounded by openings O (also referred to herein as orifices) which result from the diastolic pressure gradient. Advantageously, leaflets LF are coapted such that their proximal or upstream surfaces face each other in a vertical orientation, parallel to the direction of blood flow through the valve. The upstream surfaces may be brought together so as to be in contact with one another or may be held slightly apart but will preferably be maintained in the vertical orientation in which the upstream surfaces face each other at the point of coaptation. This simulates the double orifice geometry of a standard surgical bow-tie repair. Color Doppler echo will show if the regurgitation of the valve has been reduced. If the resulting flow pattern is satisfactory, the leaflets LF may be fixed together in this orientation. If the resulting color Doppler image shows insufficient improvement in valve regurgitation, fixation device 112 may be repositioned. This may be repeated until an optimal result is produced wherein the leaflets LF are held in place.

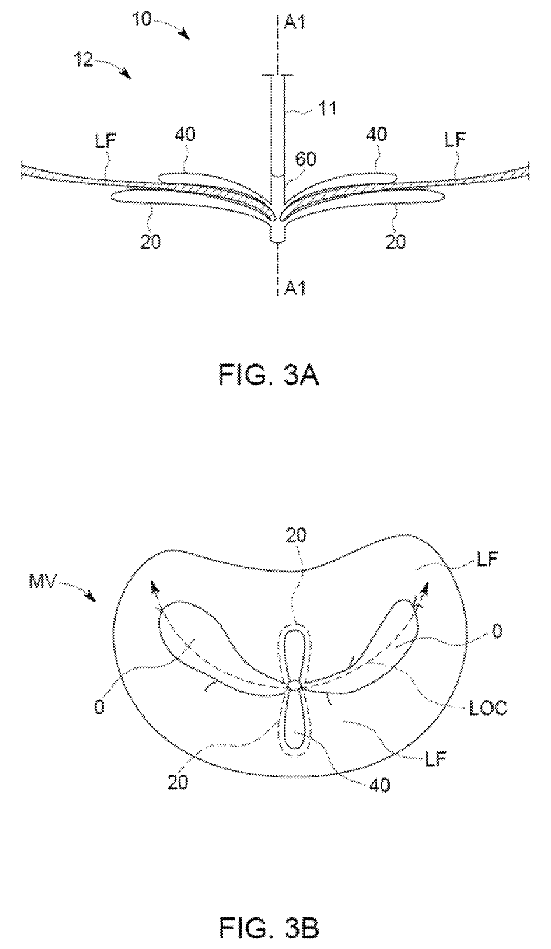

FIGS. 4A-19C depict a fixation device 112 according to another embodiment of the present disclosure. Fixation device 112 may generally include a pair of distal elements 120, a pair of proximal elements 140, a coupling member 160, an actuation mechanism 113, and a stud 131. Distal elements 120 may include elongate arms 121 in which each arm has a proximal end portion 121a, which may be rotatably connected to the coupling member 160, and a free end 121b, as best shown in FIG. 4A. Free ends 121b may each have a rounded shape to minimize interference with and trauma to surrounding tissue structures according to one example. In one example, each free end 121b defines a curvature extending about two axes 126, 127. The first axis 126 may be a longitudinal axis of each respective arm 121. Additionally, arms 121 may each include an engagement surface 125 that may also be curved about first axis 126 and may extend at least partially along a length of arm 121 to the free end 121b. Thus, in some examples, engagement surfaces 125 may each have a cupped or concave shape which may maximize contact area engagement with tissue and may assist in grasping and holding valve leaflets. Such cupped or concave shape may further allow arms 121 to nest around shaft 111 of interventional tool 110 while in the closed position to minimize the profile of device 112. Thus, arms 121 may be at least partially cupped or curved inwardly about their longitudinal axes 126 which may form a concavity extending along axis 126 which may nest proximal elements 140 when in a lowered position thereof. The second axis 127 about which each free end 121b may be curved may extend perpendicular to first axis 126, as is also shown in FIG. 4A. The curvature about this second axis 127 may be a reverse curvature located at the most distal portion of free ends 121b. In addition to the dual curvature, free ends 121b may flare outwardly at their respective longitudinal edges. It is believed that both the reverse curvature and flare help create an atraumatic configuration that minimizes trauma to the tissue engaged therewith.

In the nonlimiting embodiment depicted, a transverse width across engagement surfaces 125 (which is in the direction of second axis 127 and determines the width of tissue engaged) may be at least about 2 mm, 3-10 mm in some examples, and about 4-6 mm in some examples. In some embodiments, a wider engagement may be desired wherein the engagement surfaces 125 are larger, for example about 2 cm, or multiple fixation devices 112 may be used adjacent to each other. Arms 121 may also have a length of about 6-12 mm (defined along first axis 126), and engagement surfaces 125 may be configured to engage a length of tissue of about 4-10 mm along the longitudinal axis 126 of arms 121 according to some examples. Also, as shown in the illustrated example, each arm 121 may include a plurality of openings 128 to enhance grip and to promote tissue ingrowth following implantation.

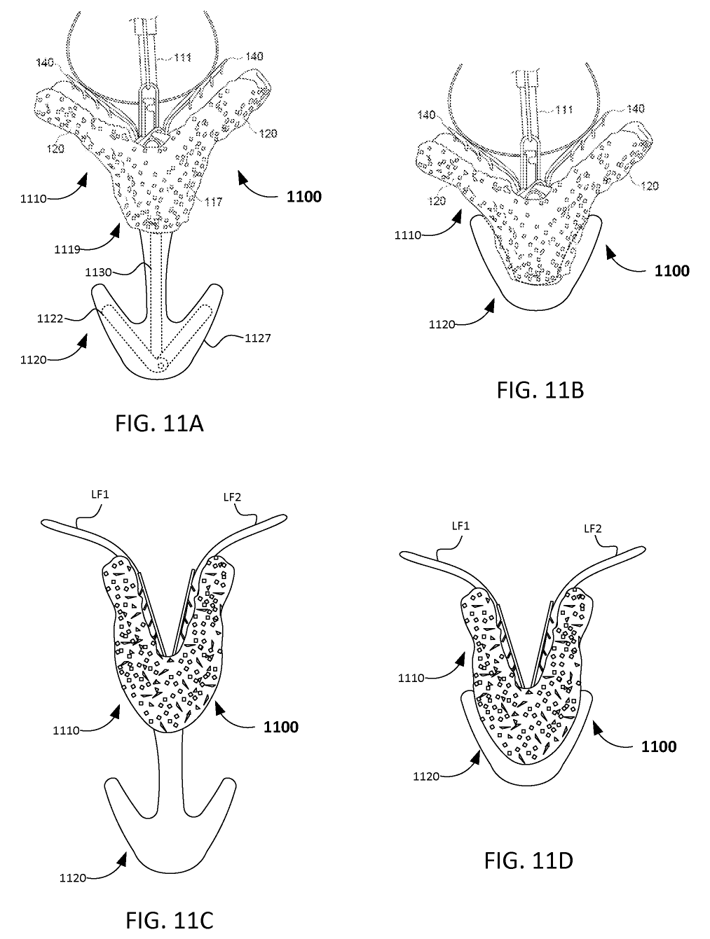

In one example, actuation mechanism 113 may include two link members or legs 130. Legs 130 may be comprised of a rigid or semi-rigid metal or polymer such as Elgiloy®, cobalt chromium or stainless steel, however any suitable material may be used. Each leg 130 may have a first end 132, which may be rotatably joined with one of the distal elements 120 at a riveted joint 135, and a second end 134, which may be rotatably joined with stud 131, as shown in FIG. 4A. Although the depicted embodiment shows both legs 130 pinned to stud 131 by a single rivet 135, it is also contemplated that each leg 130 may be individually attached to the stud 131 by a separate rivet, pin or the like. In other embodiments of actuation mechanism 113, actuation mechanism 113 may include a base 139, and second ends 134 of legs 130 may be rotatably joined with base 169, such as by one or more riveted joints 135, as best shown in FIG. 10B. An actuator rod 170 of delivery system 1000 may be joinable with actuation mechanism 113 directly, such as via direct connection with base 139, or indirectly, such as via connection with stud 131, which itself may extend from base 139. In either of these embodiments, actuator rod 170 may be axially extendable and retractable in a proximal-distal direction to actuate actuation mechanism 113 and consequently rotate distal elements 120 between open, closed, and inverted positions, which are described further below. Additionally, coupling member 160, stud 131, and/or base 169 may comprise a center portion or center body of fixation device, for example.

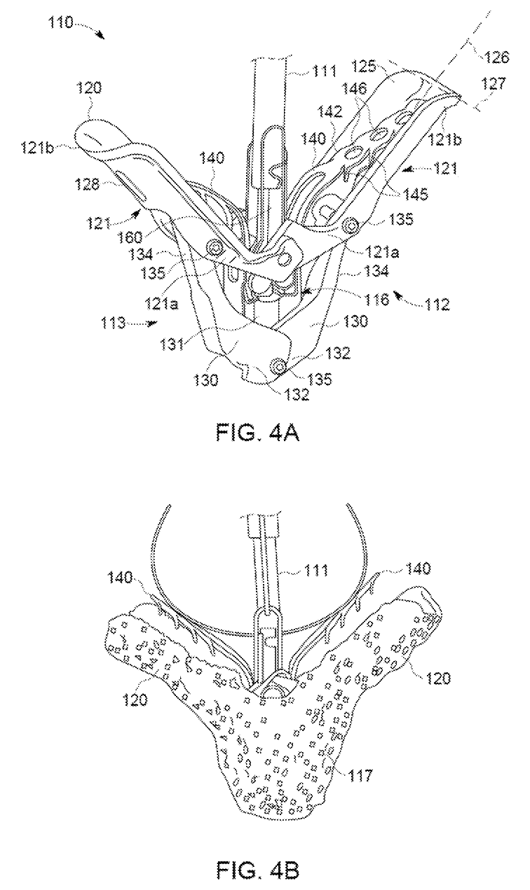

Proximal elements 140 may, in some examples, be flexible, resilient, and cantilevered from a center of fixation device 112. For example, FIGS. 5A and 5B depict a gripping device 114 according to an embodiment of the present disclosure that may generally include a pair of proximal elements 140, a base section 150, and a pair of arm bend features 153 partitioning proximal elements 140 from base section 150.

Proximal elements 140 may be in the form of elongate arms 141 that each extend along a longitudinal axis A2 from a first end portion or fixed end 141a to a second end portion or free end 141b, as shown in FIG. 5A. Each proximal element 140 may also have opposed side edges 142 that define a width transverse to the longitudinal axis A2. Such width may be less than the width of a corresponding distal element 120 such that proximal element 140 may be recessed within the concavity formed by engagement surface 125 of distal element 120 when proximal element 140 is moved into a lowered position, as described in more detail below.

Proximal elements 140 may also each have a first side or proximal side 143 and a second side or distal side 144. In one example, proximal elements 140 may include a plurality of openings 146 that may extend from proximal side 143 to distal side 144, as shown in FIG. 5A. Such openings 146 may be used to couple a proximal element line, which is discussed further below, to a proximal element 140 for raising and lowering proximal element 140. Each proximal element 140 may also include one or more frictional elements 145 extending from distal side 144. For example, each proximal element 140 may include one or more rows of frictional elements 145 where frictional elements 145 in each row may be aligned in a direction transverse to longitudinal axis A2. Frictional elements 145 in such rows may also be aligned with frictional elements 145 in other rows in a lengthwise direction thereby forming columns of frictional elements 145. For example, in the embodiment depicted in FIGS. 5A and 5B, each proximal element 145 may include four rows of two frictional elements 145. In other words, two columns of four frictional elements 145. In other embodiments, proximal elements 140 may include one to six rows of two to six frictional elements 145 per row, for example. However, in other embodiments, frictional elements 145 may be arranged in an offset relationship in a lengthwise and/or transverse direction such that at least some frictional elements 145 are not aligned with another frictional element 145 in such directions.

Frictional elements 145 may comprise frictional protrusions or tines having tapering pointed tips extending from distal side 144 of proximal elements 140. Frictional elements 145 may also be angled toward fixed end 141a of proximal element 140 which may help prevent frictional elements 145 from inadvertently snaring tissue during repositioning of fixation device 112. In one example, frictional elements 145 may be integral with or connected to a distal surface 144 of a proximal element 140 and protrude therefrom. In another example, as shown in FIG. 5A, frictional elements 145 may be formed from side edges 142, such as by cutting and bending the base material forming proximal elements 140, for example. It may be appreciated that any suitable frictional elements may be used, such as prongs, windings, bands, barbs, grooves, channels, bumps, surface roughening, sintering, high-friction pads, coverings, coatings, or a combination of these. However, it should be noted that some types of frictional elements that can be utilized may permanently alter or cause some trauma to the tissue engaged. Thus, it is preferable that frictional elements 145 be atraumatic and generally frictional rather than penetrative so as to not injure or otherwise affect the tissue in a clinically significant way.

Base section 150 may be connected to a center portion or center body of fixation device 112 such that proximal elements 140 extend outwardly therefrom. For example, base section 150 may be coupled to coupling member 160. In the embodiment depicted, base section 150 may include a first member 152, a second member 154, and a third member 156. First and third members 152, 156 may be connected to second member 154 to form a generally U-shaped or box-shaped structure which may allow a locking mechanism (discussed below) to be positioned between first and third members 152, 156. However, other shapes may be formed, such as a V-shape, a crescent shape, or semicircular, for example. In some embodiments, first and third members 152, 156 may be connected to second member 154 via base bend features 157, for example. Also, second member 154 may include an opening 158 extending therethrough for receipt of stud 131 and/or actuator rod 170, as shown in FIG. 5A.

Arm bend features 153 may couple a respective proximal element 140 and base section 150. For example, an arm bend feature 153 can couple a proximal element 140 to first member 152 of base section 150, and another arm bend feature 153 can coupled the other proximal element 140 to third member 156 of base section 150. As shown, arm bend features 153 may form a living hinge about which proximal elements 140 may bend relative to base section 150. In this regard, arm bend features 153 may be integral with proximal elements 140 and base section 150 and may bias proximal elements 140 to a relaxed position. As illustrated in FIG. 5B, proximal elements 140 may form a relaxed angle 149 formed between proximal sides 143 of each proximal element 140. Such relaxed angle 149 is formed when proximal elements 140 are in the relaxed position and may form an angle of about 85 degrees to 200 degrees (+/- 5 degrees). For example, proximal elements 140 may form a relaxed angle of 180 degrees in the relaxed position. In another example, proximal elements 140 may form a relaxed angle of 185 degrees in the relaxed position. Although the embodiment depicted illustrates bend features 153 as living hinges, in other embodiments bend features 153 may comprise a biased hinge that modularly connects proximal elements 140 to base section 150. For example, proximal elements 140 may be separately formed from base section 150 and modularly connected to base section 150 via arm bend features 153 which may each comprise a spring biased hinge biasing a respective proximal element 140 to the relaxed position, for example.

Arm bend features 153 may also each include an elongate opening extending 151 along the longitudinal axis A2 which may furcate each arm bend feature 153, as illustrated in FIG. 5A. Such an elongated opening 151 may have a uniform width extending along axis A2. However, in some embodiments, such as the embodiment depicted, elongate opening 151 may form a bowling-pin shape such that a width of opening 151 is narrower at one end (e.g., the end closest to free end 141b) than the other end (e.g., the end furthers from free end 141b) and is wider somewhere in between. Elongate opening 151 may also not be relegated to just arm bend feature 153 but may also extend from arm bend feature 153 to proximal element 140 and/or base body 150. The elongate opening 151 and corresponding furcation of arm bend features 153 may be configured (e.g., in size, shape, spacing, position, etc.) so as to provide the desired resiliency, fatigue resistance, and/or flexibility at the coinciding arm bend features 153.

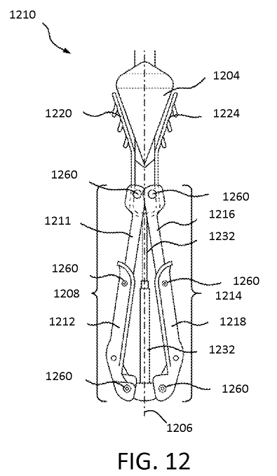

Base bend features 110 and arm bend features 112 may be configured to give gripping device 116 a bent configuration when gripping device is in a relaxed state (i.e., when proximal elements are in the relaxed position), such that when gripping device 114 is forced into a stressed state (e.g., by bending proximal elements at one or more of the base and/or arm In the exemplary embodiment depicted, gripping device 114 may be formed from a metallic sheet of a spring-like material, such as a shape-memory metal (e.g., Nitinol) which may provide the bias of proximal elements 140 toward the relaxed position. Alternatively, gripping device 114 could be molded from a biocompatible polymer. Each proximal element 112 may, in one example, be configured to be at least partially recessed within the concavity of the distal element 120 when no tissue is present. When fixation device 112 is in the open position, each proximal element 140 may be separated from the engagement surface 125 near free end 121b of arm 121 and may slope toward engagement surface 125 near free end 121b with the free end 141b of proximal element 140 contacting engagement surface 125, as illustrated in FIGS. 4A and 11B. This arrangement may be facilitated by the dimensions of base section 150. For example, increasing or decreasing the respective lengths of first, second, and third members 152, 154, 156 of base section 150 may increase or decrease the separation distance between a proximal element 140 and corresponding distal element 120 which may help accommodate a valve leaflet or other tissues of varying thicknesses. Further examples of gripping devices that may be utilized in fixation device 112 are described in more detail in U.S. Patent No. 11,096,691, the disclosure of which is incorporated by reference herein in its entirety.

Fixation device 112 may also have a covering 117, as shown in FIG. 4B. As depicted, covering 117 may encapsulate distal elements 120 and actuation mechanism 113. Thus, engagement surfaces 125 may be covered by covering 117 which may help minimize trauma on tissues and enhance primary fixation via additional friction to assist in grasping. Additionally, covering 117 on engagement surfaces 125 may facilitate tissue ingrowth to provide for secondary fixation to ensure long-term security. Covering 117 may be loosely fitted and/or may be flexible such that device 112 can freely move to various positions all the while covering 117 conforms to the contours of the device 112 and remains securely attached thereto. It may be appreciated that the covering 117 may cover specific parts of fixation device 112 while leaving other parts exposed. For example, proximal elements 140 may be exposed, while distal elements 120 and actuation mechanism 113 may be covered. However, in some embodiments, proximal elements 140 may be covered with covering 117 to enhance grip and tissue ingrowth following implantation. Preferably, when a covering 117 is used in combination with frictional elements 145 or other frictional features, such as those extending from proximal elements 140, such features may protrude through such covering 117 so as to contact any tissue engaged by proximal elements 140.

Covering 117 may be comprised of any biocompatible material, such as polyethylene terepthalate, polyester, cotton, polyurethane, expanded polytetrafluoroethylene (ePTFE), silicon, UHMWPE, or various polymers or fibers (e.g., fibers made using electrospinning) and have any suitable form, such as a fabric (woven or unwoven), mesh, textured weave, felt, looped or porous structure. Generally, covering 117 has a low profile so as not to interfere with delivery through an introducer sheath or with grasping and coapting of leaflets or tissue. Covering 117 may alternatively be comprised of a polymer or other suitable materials dipped, sprayed, coated, or otherwise adhered to the surfaces of the fixation device 112. Optionally, a polymer coating may include pores or contours to assist in grasping the tissue and/or to promote tissue ingrowth. Any of the coverings 117 may optionally include drugs, antibiotics, anti-thrombosis agents, or anti-platelet agents such as heparin, COUMADIN® (Warfarin Sodium), to name a few. These agents may, for example, be impregnated in or coated on the coverings 117. These agents may then be delivered to the grasped tissues surrounding tissues and/or bloodstream for therapeutic effects.

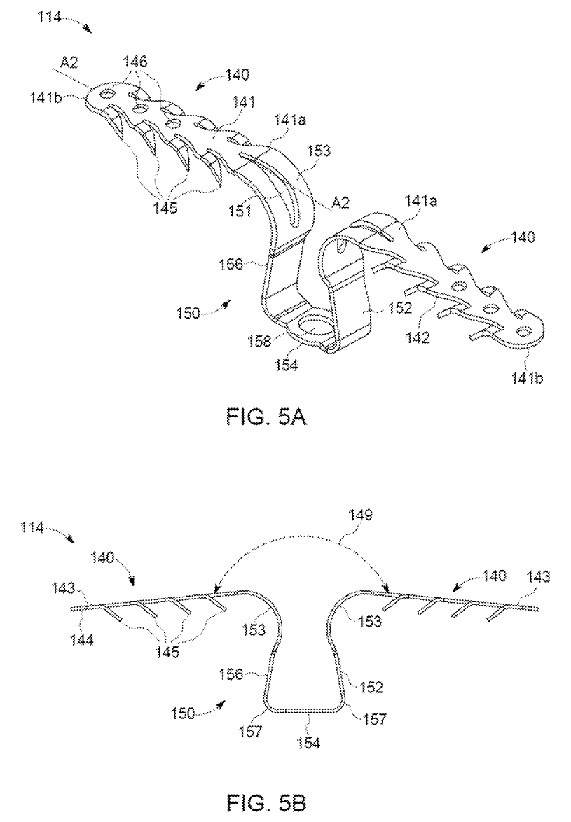

As mentioned above, fixation device 112 may, in one example, be actuated through multiple positions within a mammalian body during a transcatheter procedure such as by extending and retracting actuator rod 170 when coupled to stud 131 and/or base 139. FIGS. 6A-6B, 7A-7B, 8A-8B, 9A-9B ,and 10A - 10B illustrate several of these possible positions and in a sequence that may be utilized during a transcatheter procedure.

FIGS. 6A and 6B depict fixation device 112 in an example of a closed position or delivery position. Fixation device 112 may assume the closed position when being delivered through a guide catheter or sheath 3300 of steerable guide system 5, as shown in FIG. 6A. In the closed position, the opposed pair of distal elements 120 may be positioned so that engagement surfaces 125 thereof face each other. The cupped or concave shape of each arm 121 in this example allows arms 121 to surround shaft 111 and optionally contact each other on opposite sides of shaft 111. This provides a low profile for fixation device 112 so that it is readily passable through a delivery catheter 3300 and through any anatomical structures, such as those within the cardiovascular system.

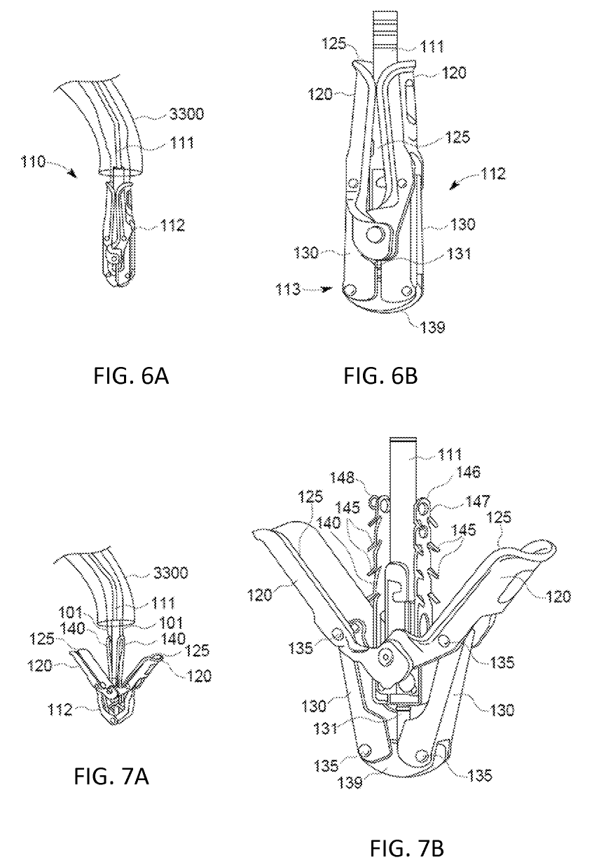

FIGS. 7A-7B depict fixation device 112 in an example of an open position. Fixation device 112 may assume the open position for capturing and grasping leaflets of a heart valve. In an open position, distal elements 120 may be rotated so that engagement surfaces 125 thereof face a first direction such that engagement surfaces 125 are disposed at an acute angle relative to shaft 111. For example, the acute angle formed between each engagement surface 125 and shaft may be 45 degrees to 90 degrees. Stated differently, in the open position, engagement surfaces 125 of distal elements 120 may be oriented 90 degrees to 180 degrees relative to each other. However, it is generally preferable for arms to be positioned 120 degrees relative to each other (and 60 degrees relative to shaft 111) for capturing leaflets. Movement of fixation device 112 from the closed position to the open position may be achieved by advancing stud 131 distally relative to coupling member 160 by distally advancing actuator rod 170. Conversely, fixation device 112 may be moved from the open position to the closed position by retracting actuator rod 170 and retracting stud 131 proximally, according to one example of the disclosure.

As shown in FIG. 7B, proximal elements 140 (or proximal elements 240) may be in a raised or insertion position when fixation device 112 is in the open position to facilitate insertion of leaflets between distal and proximal elements 120, 140 for their capture. Proximal elements 140 are, in one example, biased toward distal elements 120. In this regard, proximal elements 140 may be moved inwardly toward shaft 111 and held against shaft 111 with the aid of proximal element lines 101 which can be in the form of sutures, wires, nitinol wire, rods, cables, polymeric lines, or other suitable structures, as shown in FIG. 7A. Thus, FIGS. 7A and 10B depict fixation device 112 in an insertion configuration in which proximal elements 140 are in a raised position and distal elements 120 are in an open position.

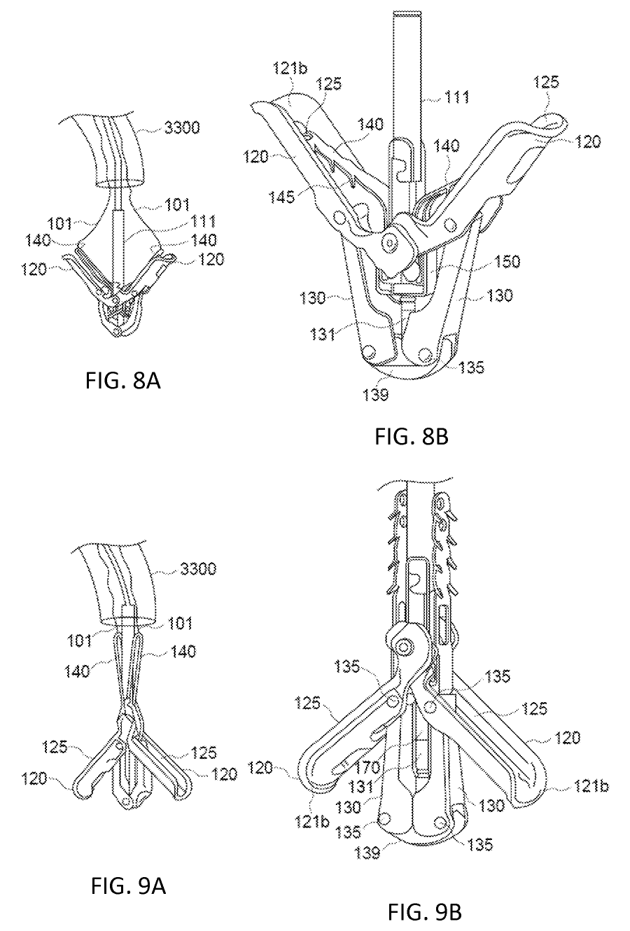

Once fixation device 112 has been positioned in a desired location against the valve leaflets, the leaflets may then be captured between proximal elements 140 and distal elements 120. FIGS. 8A and 8B illustrate fixation device 112 in an example of such a position. Here, proximal elements 140 are lowered toward engagement surfaces 125 so that proximal elements 140 are in a lowered or capture position, and the leaflets are held between distal and proximal elements 120, 140. Proximal elements 140 are, in one example, lowered into the lowered position while distal elements 120 remain in the open position. Thus, fixation device 112, as shown in FIGS. 8A and 8B is in an example of a capture configuration which may be similar to the insertion configuration of FIGS. 7A and 7B, but with the difference being that proximal elements 140 are now lowered toward distal elements 120 by releasing tension on proximal element lines 101 to compress the leaflet tissue therebetween. At any time, the proximal elements 140 may be raised and the distal elements 120 adjusted or inverted to reposition fixation device 112 if regurgitation is not sufficiently reduced according to one example of the disclosure.

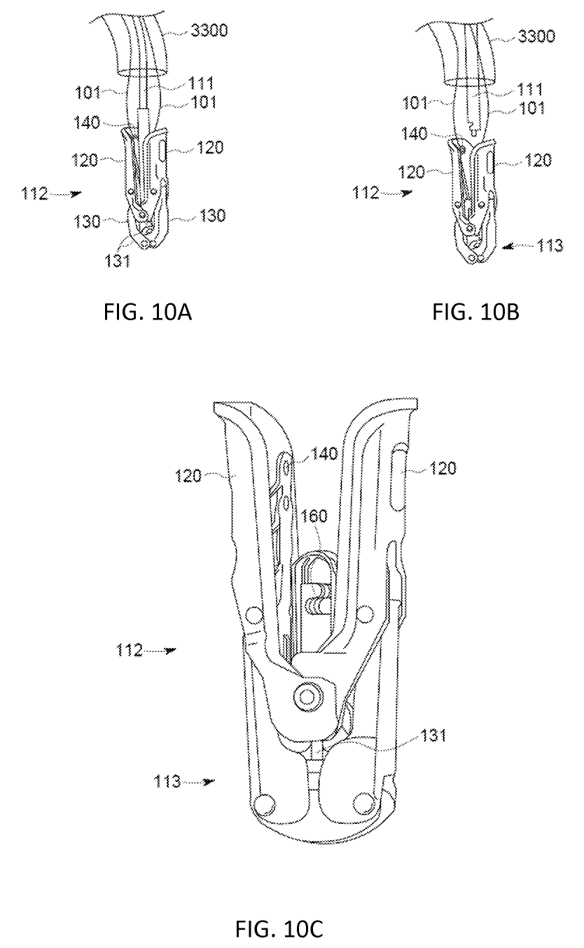

FIGS. 9A-9B depict an example of an inverted position of fixation device 112. Fixation device 112 may assume the inverted position to aid in repositioning or removal of fixation device 112. In one example of the inverted position, distal elements 120 may be further rotated from the open position, which may be achieved by advancing stud 131 further relative to the open position, so that the engagement surfaces 125 of distal elements 120 face outwardly, and free ends 121b point distally. Additionally, in some examples, engagement surfaces 125 of each arm 121 may form an obtuse angle relative to shaft 111. For example, the obtuse angle formed between each engagement surface 125 and shaft 111 may be 135 degrees to 180 degrees. Stated differently, in the inverted position, engagement surfaces 125 of distal elements 120 may be oriented 270 degrees to 360 degrees relative to each other.

Also, as shown in FIG. 9B, in one example proximal elements 140 are in their raised position against shaft 111 while distal elements 120 are in the inverted position by exerting tension on the proximal element lines 101. Thus, a relatively large space may be created between proximal and distal elements 140, 18 for repositioning. In addition, the inverted position allows withdrawal of the fixation device 112 through the valve while minimizing trauma to the leaflets. Engagement surfaces 125 provide an atraumatic surface for deflecting tissue as the fixation device is retracted proximally. It should be further noted that tines 145 of proximal elements 140 may, in some examples, be angled slightly in the distal direction (away from the free ends of the proximal elements 140), reducing the risk that tines 145 will catch on or lacerate tissue as fixation device 112 is withdrawn and while proximal elements 140 are in the raised position.

After the leaflets have been captured between distal and proximal elements 120, 140, distal elements 120 may be returned to or toward the closed position where they may be locked in place. An example of such locking is described further below. FIG. 10A illustrates fixation device 112 in the closed position wherein the leaflets (not shown) are captured and coapted. In one example, this is achieved by retraction of the stud 131 proximally relative to coupling member 160 so that the legs 130 of the actuation mechanism 113 apply an upwards force to distal elements 120 which in turn rotate distal elements 120 so that engagement surfaces 125 again face one another, similar to that of FIGS. 6A and 6B, and so that distal elements 120 rotate proximal elements 140 in a direction toward shaft 111. However, because the leaflets are captured between distal and proximal elements 120, 140, it may be desirable to keep distal elements 120 at about 20 degrees to 60 degrees relative to each other so as to limit the amount of tension and stress on the native tissue. Thus, while fixation device 112 may be returned to the closed position, such closed position may not be as closed as in the initial delivery position.

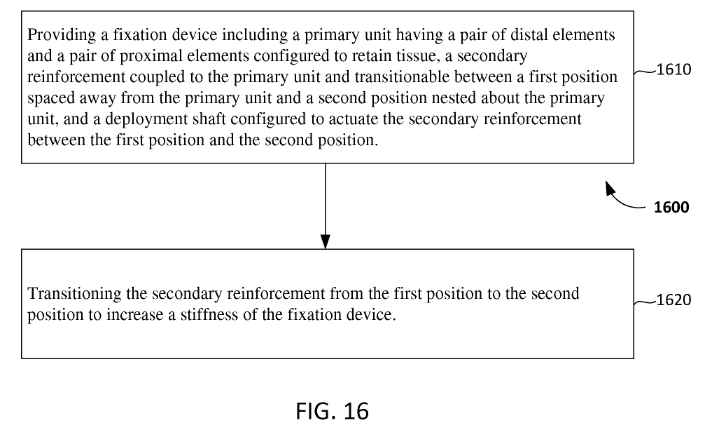

As shown in FIG. 10B, fixation device 112 may then be released from shaft 111 of delivery system 1000 while in the closed position. As mentioned, fixation device 112 may be releasably coupled to delivery system 1000 via a coupling system (e.g., coupling system 115 or 315). When the coupling structures of such coupling system are released, proximal element lines 101 may remain attached to proximal elements 140 following detachment to function as a tether to keep the fixation device 112 connected with the delivery catheter 1020 (see FIG. 16) for reconnection and repositioning. However, in other embodiments, proximal elements lines 101 may be released prior to release of fixation device 112 or concurrently with the release of fixation device 112, as described in more detail below.

FIG. 10C illustrates a released fixation device 112 in an example of a closed position. As shown, coupling member 160 remains separated from shaft 111 of delivery system 1000, and proximal elements 140 are deployed so that tissue (not shown) may reside between proximal elements 140 and distal elements 120.

In some examples, it may be beneficial to manipulate the stiffness of the implant before, during or after an edge-to-edge repair procedure. Specifically, a tunable or refinable fixation device may help users optimize the balance of mitral regurgitation or tricuspid regurgitation reduction and gradient effects by providing adjustable stiffness of the fixation device. By tunable, it will be appreciated that a fixation device may be adjusted to more than one state or position with different stiffnesses (e.g., two positions, three positions, or more). Moreover, though the disclosure refers to fixation of native leaflets, it will be understood that this is exemplary and that the systems, methods and techniques disclosed herein may be used to fix, approximate, clamp or manipulate any bodily tissue, or implanted element of a medical device (e.g., leaflets of prosthetic heart valves).

FIGS. 11A-11D illustrate one such example of a tunable stiffness fixation device 1100. It will be understood that fixation device 1100 may be similar to those previously described, and may include similar features (e.g., distal elements 120, proximal elements 140, covering 117, etc.). Those elements previously described with reference to FIGS. 10A-10C will be collectively referred to as the primary unit (or base unit) 1110. In addition to the primary unit 1110, fixation device 1100 further includes a secondary reinforcement 1120 coupled to the distalmost end 1119 of primary unit 1110 and moveable relative thereto. Secondary reinforcement 1120 may include a pair of arms 1122 coupled together (e.g., pivotably coupled) and releasably coupleable to a deployment shaft 1130. Alternatively, secondary reinforcement 1120 may be formed of a single U-shaped or V-shaped arm that opens toward the primary unit 1110 and couples to the deployment shaft 1130 at its apex. In some examples, deployment shaft 1130 is centrally located so as to bisect primary unit 1110 and/or reinforcement 1120.

In the example shown in FIGS. 11A-11D, secondary reinforcement 1120 is covered by a secondary covering 1127 similar to covering 117 to enhance grip and tissue ingrowth following implantation. Secondary reinforcement 1120 may be transitionable between two positions including a first position laterally spaced away from primary unit 1110, and specifically distal elements 120 of the primary unit (FIG. 11A), and a second position disposed closer to, or contacting, primary unit 1110, and specifically distal elements 120 of the primary unit (FIG. 11B). Actuating secondary reinforcement 1120 between the two positions may be effected by pulling on deployment shaft 1130 to bring the secondary reinforcement 1120 closer to the primary unit 1110 or pushing the deployment shaft 1130 to move the secondary reinforcement 1120 away from the primary unit 1110. Optionally, instead of being manipulated through deployment shaft 1130, an external control (e.g., pull cable) may be used to actuate secondary reinforcement 1120 independently through an action of the user of the device. In the first position, fixation device 1100 may function as previously described and primary unit 1110 may have a first stiffness, the secondary reinforcement 1120 providing no impact on the performance of the primary unit. In the second position, primary unit 1110 (e.g., distal element 120) may be nested within secondary reinforcement 1120, and fixation device 1100 may have a composite stiffness that is greater than the first stiffness of primary unit 1110 in the first position, secondary reinforcement 1120 reinforcing primary unit 1110 to increase the clamping force of fixation device 1100 onto a target (e.g., leaflets or other tissue). Thus, in this example, the effective stiffness of fixation device 1100 is selectable by the user by advancing or retracting secondary reinforcement 1120 with respect to primary unit 1110 so that the secondary reinforcement serves as extra support around the primary unit (e.g., distal elements 120).

It will be understood that secondary reinforcement 1120 may comprise a different and/or stiffer material compared to primary unit 1110. Additionally, secondary reinforcement 1120 may comprise a same material (e.g., nitinol) as primary unit 1110, but may include a different (e.g., lower) austenite finish transformation temperature relative to the primary unit 1110 to provide increased stiffness. In some examples, secondary reinforcement 1120 may have a thicker cross-section or increased moment of inertia compared to the primary unit 1110. In some examples, secondary reinforcement 1120 may have a shorter beam length attachment compared to the primary unit 1110. These techniques (e.g., differences in material, finishing transformation temperature, cross-sections, beam length attachment, etc.) may be combined as desired. Additionally, any of the embodiments may be adapted to increase the surface area of contact with tissue (e.g., to increase effectiveness) or may be nested fully within the extent of the surface area of contact with tissue (e.g., to maintain gradient without increasing effectiveness) when reinforcement is applied.

FIGS. 11C-11D show the fixation device 1100 in two fully implanted positions. In FIG. 11C, fixation device 1100 is being used to capture leaflets LF1,LF2 between proximal elements 140 and distal elements 120, secondary reinforcement 1120 being spaced away from the primary unit. In this implanted position, primary unit 1110 may be configured to have a first stiffness S1, which may provide a reduced reduction of mitral or tricuspid regurgitation but may also reduce the amount of flow obstruction in the repaired valve (i.e., less gradient increase after implantation).

In some examples, in the closed state, distal elements 120 of primary unit 1110 may begin to deflect outward when pulled apart at its tip with a stiffness of between 0.8 and 2.0 lbf per inch, or between 1.0 and 1.4 lbf per inch or approximately 1.2 lbf per inch, and this may represent first stiffness S1. Stiffness may be measured in various ways, and in some examples, stiffness may be measured by pulling the distal elements 120 at the midpoint of each arm at a crossbar feature to estimate stiffness. In other examples, it may be possible to estimate or measure stiffness by pulling from the distal element tips. In some examples, the distal elements may open from about 15 degrees to about 45 degrees when a particular force (e.g., 1.2 lbf) is applied.

FIG. 11D shows fixation device 1100 being used to capture leaflets LF1,LF2 between proximal elements 140 and distal elements 120, secondary reinforcement 1120 being nested on, or clamped about, primary unit 1110. In this implanted position, fixation device 1100 may be configured to have an increased composite second stiffness S2. In some examples, second stiffness S2 stiffness may be between 1.6 and 4.0 lbf per inch. Second stiffness may also be 100%, 200%, 300%, 400% greater than first stiffness S1. Second stiffness may also be more than 400% greater than first stiffness S1. In some examples, second stiffness S2 may be between 15-25 lbf./inch.

In some examples, composite second stiffness S2 may be 50% greater than first stiffness S1. In some examples, composite second stiffness S2 may be 100%, 150%, 200%, 250%, 300%, 350%, or 400% greater than first stiffness S1. Second stiffness S2 may reduce mitral or tricuspid regurgitation even further, but may also increase the amount of flow obstruction in the repaired valve (i.e., higher gradient after implantation). Thus, the stiffness of the implant may directly affect the amount of annulus diameter reduction achieved by the implant (i.e., the “annuloplasty effect” on the valve annulus). For example, a stiffer device (e.g., two-times stiffer or higher) may better stabilize the annulus, but may also reduce its size more than a more flexible device. Because of the tradeoffs described above a user-selectable “in situ” implant stiffness according to the present disclosure may be possible where a user can adapt the installed fixation device to the response of the target valve. Because this response is difficult to predict, real-time implant adjustment may be made so that the user can implant a fixation device, observe performance, and take additional corrective measures if mitral or tricuspid regurgitation reduction is insufficient (e.g., by stiffening the implant with the secondary reinforcement), or if gradient has exceeded a predetermined threshold (e.g., 5 mmHg). These corrective measures may take the form of the operator adding or removing the secondary reinforcement to make the implant more rigid or more flexible, depending on the need.

Thus, by having a fixation device with multiple states or positions, the operator may choose a setting that is best suitable for each patient. Additionally, it will be understood that a fixation device may be fully or partially implanted in a first position, such as in FIG. 11C, and that a secondary reinforcement 1120 may be deployed onto the primary unit 1110 after evaluating the performance of the fixation device in the first position. The opposite is also true. A fixation device 1100 may be first implanted in the second, stiffening position, and the operator may choose to go to lower stiffness position by moving the secondary reinforcement 1120 away from the primary unit.

The construction of the primary unit of the fixation device may vary. For examples, instead of the hinged distal elements 120 described above, a fixation device may include spring-like elements or shape memory materials, such as a shape-memory metal (e.g., Nitinol) which may provide the bias to open and close the fixation device. Examples of these constructions are found in U.S. Patent Serial No. 17/484,203 the content of which is hereby incorporated by reference in its entirety as if fully set forth herein.

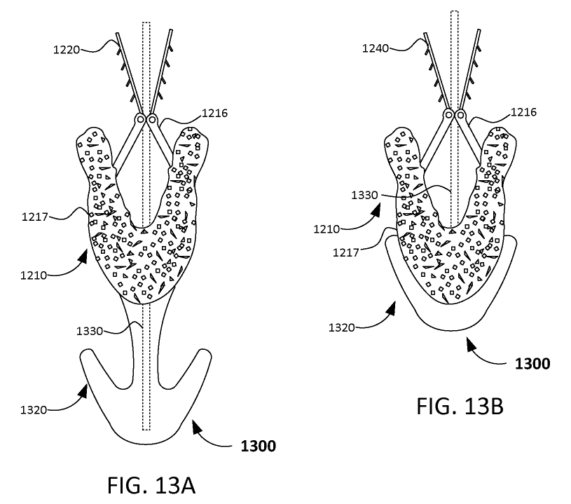

Referring to FIG. 12, for the purpose of illustration and not limitation, a different construction of primary unit 1210 will be briefly described. Primary unit 1210 may include a center portion 1204 defining a longitudinal axis 1206, a first distal assembly 1208 comprising a first distal strut 1211 pivotally-coupled with the center portion 1204 and a first distal element 1212 pivotally-coupled with the first distal strut 1211. The first distal assembly 1214 is configured to move between a closed position with the first distal strut 1211 folded proximate the center portion 1204 and the first distal element 1212 folded proximate the first distal strut 1211, and an extended position with the first distal strut 1211 extending distally from the center portion 1204 and the first distal element 1212 extending distally from the first distal strut 1211. The first distal assembly 1208 is biased towards the closed position. Primary unit 1210 further includes a second distal assembly 1214 comprising a second distal strut 1216 pivotally-coupled with the center portion 1204 and a second distal element 1218 pivotally-coupled with the second distal strut 1211. The second distal assembly 1208 is configured to move between a closed position with the second distal strut 1216 folded proximate the center portion 1204 and the second distal element 1218 folded proximate the second distal strut 1216, and an extended position with the second distal strut 1216 extending distally from the center portion 1204 and the second distal element 1218 extending distally from the second distal strut 1216. Furthermore, the second distal assembly 1214 is biased towards the closed position.

As embodied herein, each of the first and second distal assemblies 1208, 1214 can comprise a plurality of stamped metal components having at least one rivet attachment 1260. For example, the distal elements 1212, 1218 and distal struts 1211, 1216 may be stamped metal components with various rivet attachment 1260 connections, as shown for purpose of illustration and not limitation in FIG. 12. Additionally, or alternatively, each of the first and second distal assemblies 108, 114 can be made of a single-piece, braided structure. As another example, each of the first and second distal assemblies can be made of laser-cut metallic sheet structures or nitinol shape-set structures.

The extended position can be a fully inverted position of the distal elements 1212, 1218 configured for delivery of the primary unit and further configured for complete leaflet release. The first and second distal assemblies 1208, 1214 can be configured to capture a leaflet at various positions between the closed position and the extended position, such as when the angle between the first distal element 1212 and the second distal element 1218 is between about 90 and 130 degrees or between about 110 and 130 degrees, and preferably about 120 degrees. The primary unit 1210 may further include a first proximal element 1220 having a first gripping portion, wherein the first gripping portion is moveable relative to the first distal assembly 1208 to capture native leaflet tissue therebetween. Likewise, the primary unit 1210 may further includes a second proximal element 1224 having a second gripping portion, wherein the second gripping portion is moveable relative to the second distal assembly 1214 to capture native leaflet tissue therebetween. Each of the first and second proximal elements 1220, 1224 can be attached to the center portion 1204. As embodied herein, each proximal element 1220, 1224 can includes a plurality of friction elements, for example in rows. The friction elements can allow for improved tissue engagement during leaflet capture. In leaflet capture, each first and second proximal element 1220, 1224 can be lowered or moved toward each respective first and second distal assembly 1208,1214 and into contact with the atrial side of the native leaflet to capture the leaflet therebetween.

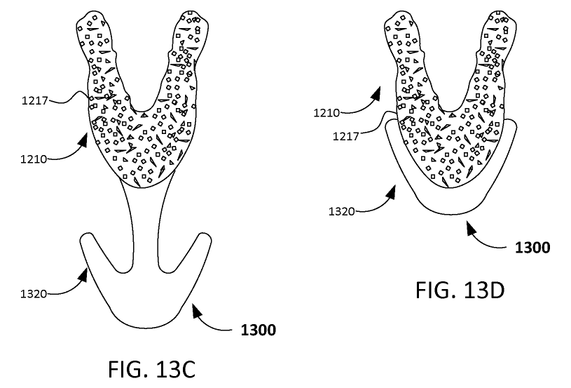

As shown in FIGS. 13A-13B, a fixation device 1300 may include primary unit 1210 of FIG. 12 being (shown here mostly concealed by covering 1217) coupled to a secondary reinforcement 1320, the secondary reinforcement 1320 being actuatable by deployment shaft 1330. As shown in FIGS. 13A-13B, the secondary reinforcement 1320 does not contact the native leaflets but is placed distal to the primary unit so that the primary unit 1210 and secondary reinforcement 1320 can be actuated in series. The relative stiffness of the primary unit and the secondary reinforcement may be tuned to be controllable in series using deployment shaft 1330 as previously described. It will be note that in some examples, deployment shaft 1330 may be the same shaft that actuates the primary unit 1210. Deployment shaft 1330 may, for example, control the primary unit and/or secondary reinforcement 1320 by distally pushing forward or retracting proximally to actuate secondary reinforcement 1320. Alternatively, secondary reinforcement 1320 may be actuated by a second, independent actuator shaft threaded to base of secondary reinforcement 1320 that is separate from the shaft that actuates the primary unit 1210. The two shafts may be concentric or extend in a side-by-side configuration. Thus, a single shaft may be used to actuate both the primary unit and the secondary reinforcement, or two independent mechanically-linked or mechanically-isolated shafts may be used to actuate the primary unit 1210 and the secondary reinforcement 1320. While FIGS. 13A-13B show one example of a system used to actuate fixation device 1300, it will be understood that variations are possible and that a fixation device 1300 according to FIGS. 13C-13D may be deployed or actuated using other mechanisms.

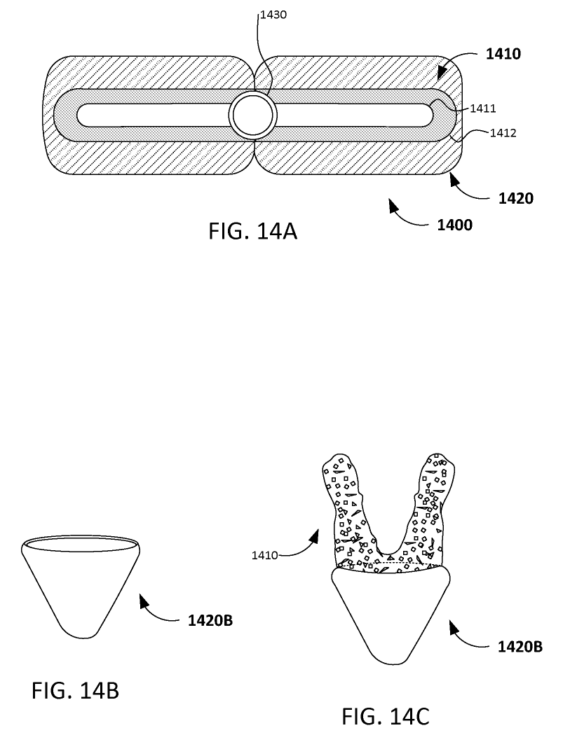

In addition to providing the ability to select fixation device stiffness, the secondary reinforcement may be used to increase the contact area with the leaflet. As shown in FIG. 14A, a fixation device 1400 may include a primary unit 1410 that includes a pair of proximal elements 1411 and a pair of distal elements 1412. Primary unit 1410 may be narrower or smaller than a corresponding secondary reinforcement 1420. For example, reinforcement 1420 may have an upper surface with a surface area that is 10%, 20%, 30%, 40%, 50% or more larger than the surface area of primary unit 1410 (e.g., of distal elements 1412). In some examples, reinforcement 1420 is longer than distal elements 1412. In some examples, reinforcement 1420 is wider than distal elements 1412. Thus, when secondary reinforcement 1420 is deployed, both the stiffness and the size of the implant’s contact with the leaflet tissue may be increased. By doing so, a single implant size with a primary unit and a secondary reinforcement may take the place of multiple distinct implant sizes, offering the user even more control and balance between regurgitation reduction and gradient effects caused by the implantation of the fixation device.

While the secondary reinforcement has been described as having a substantially U-shaped or V-shaped cross-section, it will be understood that other configurations are possible. For example, FIG. 14B shows a secondary reinforcement 1420B that is substantially cone-shaped configured to entirely surround the primary unit. In this example, secondary reinforcement 1420B is shown as being continuous along its circumference, but it is contemplated that the secondary reinforcement 1420B may be circumferentially discontinuous. FIG. 14C shows reinforcement 1420B surrounding a primary unit 1410. In some examples, a reinforcement may have flexible flaps (e.g., maybe similar to a fabric or leaflet) that open during the diastolic phase in the direction of blood flow (i.e., from the atrium to ventricle) but during the systolic phase will wrap around the fixed clip to provide additional closure/coaptation to reduce regurgitation even further.

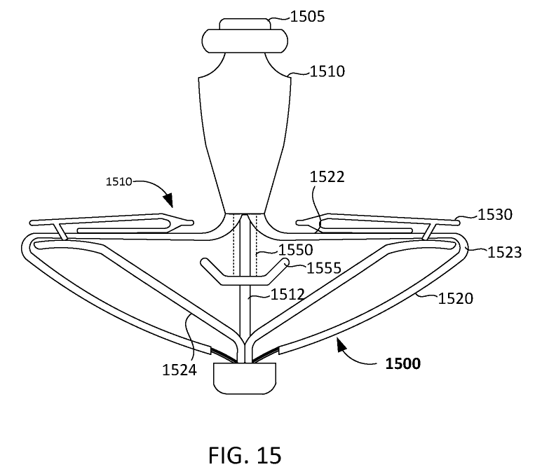

In yet another example, shown in FIG. 15, a fixation device 1500 is shown which uses inner paddles 1522, and outer paddles 1520 that move together using a single actuation wire 1512, and a pair of clasps 1530 are directly linked to the inner paddles 1522 to retain the native leaflets. Actuation wire 1512 may pass through the attachment portion 1505 and the coaption element 1510 of fixation device 1500. In this example, a secondary reinforcement 1555 may be disposed between the outer and inner paddles 1520,1522 and may be actuatable via a hypotube 1550 to optionally add stiffness to the fixation device 1500 by contacting, pressing on, or clasping inner paddle 1522. This example may be referred to as having an embedded secondary reinforcement because the reinforcement is disposed between the inner and outer paddles, and will not be visible when the fixation device is wrapped with a covering. Hypotube 1550 may independently translate distally or proximally along the longitudinal axis of the fixation device and may pull secondary reinforcement 1555 therewith so that the fixation can transition between two positions (i.e., a first position with the secondary reinforcement 1555 providing stiffness to the inner paddle and a second position with the secondary reinforcement 1555 being spaced away from the inner paddle). In some examples, the reinforcement being applied may increase the stiffness incrementally (e.g., 200%) and/or be applied to increase stiffness by an order of magnitude (e.g., by a factor of 10or more), effectively "locking" the device or making it effectively rigid. Different reinforcement "V" members may be used to rigidly lock the device at different desired angles, and these support Vs may be user-selectable before implantation based on the need for rigidity depending on the anatomy or disease being treated. For example, more rigidity could be needed for degenerative disease with large flailing leaflets.

FIG. 16 is a flowchart showing one example of a method 1600 according the present disclosure. The method 1600 may include a first step 1610 of providing a fixation device including a primary unit having a pair of distal elements and a pair of proximal elements configured to retain tissue, a secondary reinforcement coupled to the primary unit and transitionable between a first position spaced away from the primary unit and a second position nested about the primary unit, and a deployment shaft configured to actuate the secondary reinforcement between the first position and the second position. The method may include a second step 1620 of transitioning the secondary reinforcement from the first position to the second position to increase a stiffness of the fixation device.

It is to be understood that the fixation devices and components thereof described above are provided as examples are not to be considered as limiting to fixation devices suitable for use with other aspects of the disclosure. Additionally, it will be understood that the primary unit and/or secondary reinforcement 1420 are controllable in-situ by the user. Alternatively, in some examples, manual configuration of the secondary reinforcement 1420 is also possible on the bench (e.g., pre-surgery or prior to introducing the device into the patient). Moreover, additional sets of reinforcements 1420 (e.g., three, four or more sets) may be used and the sets may be nested within one another for greater range of tunability. In some variations, structural elements like springs are added in series or in parallel to provide support alongside the primary unit. Another way to increase or adjust stiffness of an implant's support structure is to employ hollowed structures that can be filled with fluid or curable polymer. Another approach is to employ central support structures (like a V-shape) that start as slender long telescoped out V-arms that can be pulled back inward to increase the cross section near the central support. This may reduce the length of the support arms but increases the bending stiffness, especially if the outer shell cross section with highest moment of inertia is pulled back to cover the more central smaller structure. An example would be a telescoping V-arm support configuration that initially is 3x long and slender on both sides (~12 mm), that has its distant members pulled back to translate and stack into itself at 1/3 of its original length (~4mm), which creates a stubby but support bending support similar to element 1555 in FIG. 15.

Although the subject matter disclosed herein has been described with reference to particular embodiments, it is to be understood that these embodiments are merely illustrative of the principles and applications set forth in this disclosure. It is therefore to be understood that numerous modifications may be made to the exemplary embodiments and that other arrangements may be devised, such as combining one or more features of one embodiment with another embodiment or features from a plurality of embodiments, as an example. Thus, the exemplary embodiments herein are not intended to be exhaustive or to limit the disclosed subject matter to such embodiments.

1. A fixation device comprising:
a primary unit having pair of distal elements and a pair of proximal elements, the pair of distal elements and the pair of proximal elements being configured to retain tissue;
a secondary reinforcement coupled to the primary unit and transitionable between a first position spaced away from the primary unit and a second position nested about the primary unit; and
a deployment shaft configured to actuate the secondary reinforcement between the first position and the second position.

2. The fixation device of claim 1, wherein the deployment shaft controls both the primary unit and the secondary reinforcement.

3. The fixation device of claim 1, wherein the primary unit has a first stiffness when the secondary reinforcement is in the first position, and the fixation device has a second composite stiffness when the secondary reinforcement is in the second position, the second stiffness being greater than the first stiffness.

4. The fixation device of claim 1, wherein the secondary reinforcement has an upper surface with a surface area larger than the upper surface of the primary unit.

5. The fixation device of claim 1, wherein the secondary reinforcement is longer or wider than the distal elements of the primary unit.

6. The fixation device of claim 1, further comprising additional sets of reinforcements nestable about the secondary reinforcement.

7. The fixation device of claim 1, wherein the secondary reinforcement includes a pair of arms pivotably connected to one another and to the deployment shaft.

8. A fixation device comprising:
a primary unit having pair of distal elements and a pair of proximal elements, the pair of distal elements and the pair of proximal elements being configured to retain tissue;
a secondary reinforcement coupled to the primary unit and transitionable between a first position spaced away from the primary unit and a second position clamping about the primary unit to provide increased stiffness; and
a deployment shaft configured to actuate the secondary reinforcement between the first position and the second position.

9. The fixation device of claim 8, wherein the deployment shaft controls both the primary unit and the secondary reinforcement.

10. The fixation device of claim 8, wherein the deployment shaft controls only the secondary reinforcement and further comprising an independent primary unit shaft configured to actuate the primary unit.

11. The fixation device of claim 8, wherein the secondary reinforcement has an upper surface with a surface area larger than the upper surface of the primary unit.

12. A method of treating a native valve comprising:
providing a fixation device including a primary unit having a pair of distal elements and a pair of proximal elements configured to retain tissue, a secondary reinforcement coupled to the primary unit and transitionable between a first position spaced away from the primary unit and a second position nested about the primary unit, and a deployment shaft configured to actuate the secondary reinforcement between the first position and the second position.

13. The method of claim 12, further comprising transitioning the secondary reinforcement from the first position to the second position to increase a stiffness of the fixation device.

14. The method of claim 12, further comprising actuating both the primary unit and the secondary reinforcement via the deployment shaft.

15. The method of claim 13, wherein transitioning the secondary reinforcement to the second position increases a surface area of the fixation device in contact with the tissue.

16. The method of claim 13, wherein transitioning the secondary reinforcement from the first position to the second position brings the secondary reinforcement closer to the primary unit or wherein transitioning the secondary reinforcement from the first position to the second position comprises clamping the secondary reinforcement onto the primary unit.

17. The method of claim 13, further comprising implanting the fixation device to retain native valve leaflets and assessing a performance of the fixation device prior to transitioning the secondary reinforcement from the first position to the second position or further comprising implanting the fixation device to retain native valve leaflets after transitioning the secondary reinforcement from the first position to the second position.

18. The method of claim 13, further comprising transitioning the secondary reinforcement from the second position to the first position to reduce the stiffness of the fixation device.

19. The method of claim 13, further comprising nesting the secondary reinforcement around at least a portion of the primary unit.

20. The method of claim 13 further comprising:
examining performance of the fixation device; and
actuating the secondary reinforcement so that the secondary reinforcement and the primary unit at least partially overlap with one another.
